(19) United States
(12) Patent Application Publication
(10) Pub. No.: US 2003/0204192 A1
(43) Pub. Date: Oct. 30, 2003

(54) System for hair removal

(76) Inventor: Micheline Schulte, Chandler, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/083,855

(22) Filed: Feb. 27, 2002

(51) Int. Cl. A61M 35/00; A47L 13/30; B43M 11/06; B43K 5/00; A61B 17/50

(52) U.S. Cl. 606/133; 401/261; 604/289; 604/290

(57) ABSTRACT

A system for hair removal including a hair removal solution, a first pad for applying the hair removal solution, and a package enclosing the first pad in a first inner pouch is disclosed. The disclosed system may also include a second inner pouch, opposite to the first inner pouch, in which a second pad or additional hair removal solution may be enclosed. A method for making the system for hair removal and a method for making the hair removal solution are also disclosed. The hair removal system as described herein is a convenient, painless, non-irritating, compact, portable, disposable, easy-to-use, all-encompassing system for removing hair. The user simply opens the package, removes the pad, applies the hair removal solution for the prescribed time, and then removes the hair and residue with the cleaning pad or uses a towel or water.

### FIELD OF THE INVENTION

The present invention relates to a system for hair removal and, more particularly, to a system utilizing a pad containing a hair removal solution enclosed within a sealed package.

### BACKGROUND OF THE INVENTION

It is common for men and women to seek removal of unwanted hair to improve personal appearance. Men remove hair from ears, between the eyebrows, and the back. Women remove unwanted facial, underarm, leg, and bikini hair.

The most popular method of hair removal is shaving. Though shaving is advantageous because the shaving equipment is generally portable, lightweight, and inexpensive, shaving also presents several disadvantages such as cuts from the razor and pain. Another lightweight and inexpensive alternative for hair removal is tweezing. Tweezing causes pain and irritation as well.

Waxing has also been popular. Men and women have used waxes to remove hair. The wax is warmed to a liquid and then applied to the area presenting undesired hair. The wax is then cooled and hardened, embedding the hairs into the wax. The user then strips away the wax, thereby removing the hair. Such methods may be time-consuming and messy. Additionally, stripping the hair from the body parts may cause pain and irritation.

The least painful method of hair removal is use of creams or lotions. These creams or lotions contain thioglycolates, which work by hydrolyzing disulfide bonds between the cystine molecules of the hair strand. Modern formulations combine thioglycolates with alkali such as sodium hydroxide or calcium hydroxide. Thus, not only will the disulfide bonds of the hair break, but an additional hydration of the hair shaft occurs causing the hair to become jelly-like.

Just as with waxing, use of creams or lotions may be time-consuming and messy. A user must open a bottle and self-apply the lotion or cream with a tissue or cotton ball to the area with undesired hair. During travel, the bottle may open or break and spill hair removal solution onto clothes or personal belongings. A traveler may forget to bring tissue or cotton balls making it inconvenient to apply the hair removal lotion or cream.

Moreover, due to the increases in security awareness, many airports do not allow tweezers or razors in carry-on luggage. The security measures pose a problem for those travelers who want to travel light. Travelers who inadvertently bring tweezers or razors in carry-on luggage inevitably end up relinquishing possession of the items and purchasing new hair removal equipment at their destination.

Therefore, there is a need for a safe, simple, inexpensive, and novel system for hair removal. Furthermore, there is a need for a system that will not be suspect when passing through security of airports or likely to be turned over to authorities during travel.

### BRIEF DESCRIPTION OF THE DRAWINGS

FIG. 1 is a cut-away view of an exemplary embodiment of the hair removal system;

FIG. 2 illustrates a front view of a second embodiment of the hair removal system; and

FIG. 3 illustrates a back view of the second embodiment of the hair removal system.

### DETAILED DESCRIPTION OF THE DRAWINGS

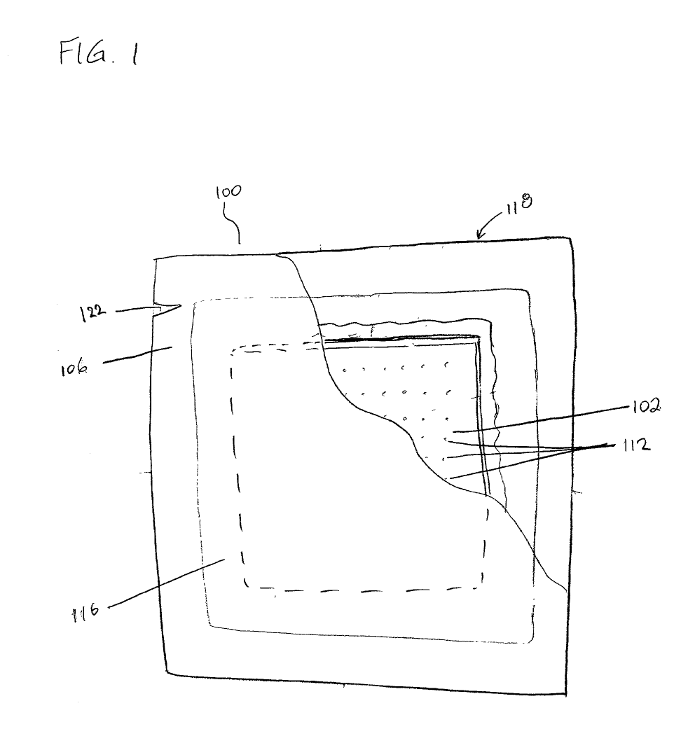

A cut-away view of hair removal system 100 is depicted in FIG. 1. As illustrated, hair removal system 100 includes pad 102 containing a hair removal solution. Pad 102 is enclosed within a sealed package 106.

Pad 102 has, for example, a length and width of about 3.0-6.0 centimeters on each side and a thickness of about 1.0-3.0 millimeters, and includes first and second major surfaces.

The size of pad 102 may be varied depending upon the amount of hair removal solution in pad 102 and for the area from which the hair is to be removed. For example, a pad for removing facial hair may be smaller than a pad for removing leg hair. The first and second major surfaces of pad 102 include a plurality of pores or indentations 112 embedded within the first and second major surfaces and extending into a middle portion of pad 102. Pad 102 is a porous material made of any one or combination of numerous materials such as felt, sponge, gauze, cotton, terry cloth material, or other natural or synthetic material capable of containing the hair removal solution. Alternatively, pad 102 may be a non-porous material. Pad 102 can be shaped into a number of shapes such as square, round, rectangle, polygonal or oval.

The plurality of pores 112 are useful for impregnating, trapping, containing, and holding the hair removal solution within pad 102 with hair removal solution. Accordingly, pad 102 is saturated with the hair removal solution. The hair removal solution may be a liquid preparation such as a lotion or a cream, or any other preparation designed for application to the surface of skin.

As depicted in FIG. 1, pad 102 is enclosed and sealed within an inner pouch of package 106. A single pad 102 is shown for hair removal system 100, but in another embodiment, more than one pad 102 may be contained within package 106. Pad 102 can be a solid patch or a folded cloth which unfolds into a larger area for use. For efficient use of materials, the inner pouch of package 106 is made substantially the same size as pad 102. Package 106 comprises a first piece 116 and a second piece 118, which are constructed from any one of numerous materials including plastic, paper, or combination thereof, so as to prevent or limit evaporation of the hair removal solution and act as a barrier to prevent or limit the hair removal solution from soaking through to the first piece 116 or second piece 118. First piece 116 and second piece 118, as shown in FIG. 1, are sized with substantially the same dimensions and the edges of first 116 and second 118 pieces are collocated with one another. Once lined up, the edges of first 116 and second pieces 118 are brought together and fused or joined around the perimeter with any one of numerous methods including the use of adhesive, glue, sealant, or melting plastic to form the inner pouch.

As shown in FIG. 1, sealed package 106 includes a perforation or cut portion 122 so that a user can easily and conveniently unseal package 106 by tearing away a portion of package 106, beginning with perforation portion 122, sufficient to readily remove pad 102.

Sealed package 106 may also contain additional hair removal solution that flows freely within the inner pouch. After initial use of pad 102, the user can dab or soak up the additional hair removal solution still contained with the inner pouch with pad 102 to continue the application process and increase the area of coverage with hair removal system 100.

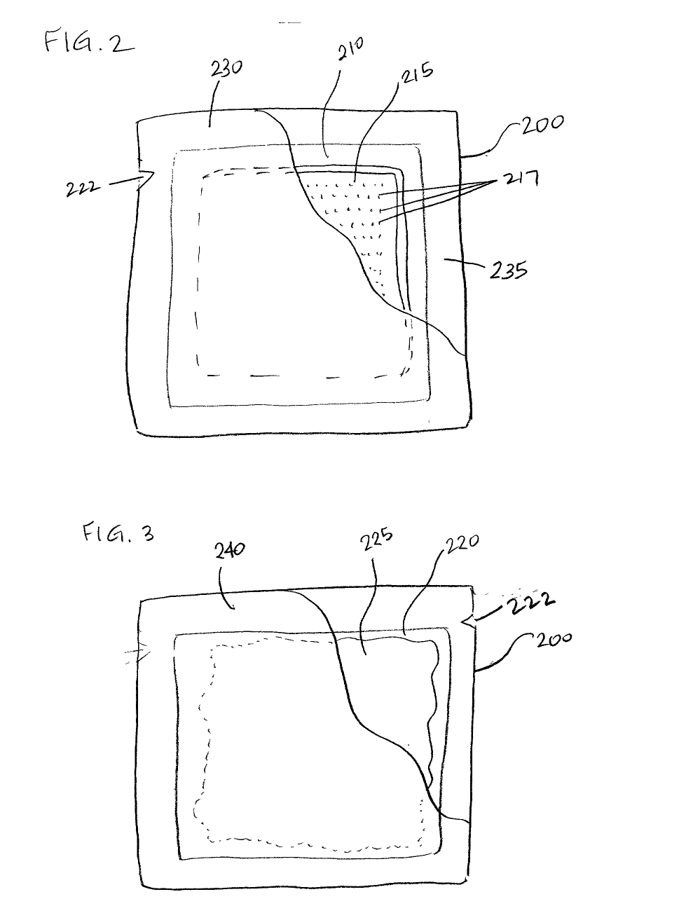

Another embodiment of the hair removal system is shown in FIGS. 2 and 3 including a package 200 which is constructed with first 230, second 235, and third 240 pieces of material fused or joined together to form a first pouch 210 on one side of package 200 as shown in FIG. 2 and a second pouch 220 on the opposite side of package 200 as shown in FIG. 3. The first 230, second 235, and third 240 pieces of material are made from plastic, paper, or combination thereof, so as to prevent or limit evaporation of the hair removal solution and act as a barrier to prevent or limit the hair removal solution from soaking through any of the materials. The first piece of material 230 and the second piece of material 235 are sized with substantially the same dimensions. The edges of first piece 230 and second piece 235 are collocated and fused or joined around the perimeter using an adhesive, glue, sealant, or melted plastic to form first pouch 210. The second piece of material 235 and third piece of material 240 are also sized with substantially the same dimensions. The edges of second piece 235 and third piece 240 are collocated and fused or joined around the perimeter using an adhesive, glue, sealant, or melted plastic to form second pouch 220.

In FIG. 2, a pad 215, having similar features as pad 102 in FIG. 1, is sealed within first pouch 210. Pad 215 is made of a porous material such as felt, sponge, gauze, cotton, terry cloth material, or other natural or synthetic material capable of being impregnated with the hair removal solution. Pad 215 includes a plurality of pores or indentations 217 embedded within the first and second major surfaces and extending into a middle portion of pad 215. Pad 215 is either dry or impregnated with the hair removal solution.

In FIG. 3, second pouch 220 is filled with hair removal solution 225. If pad 215 is initially dry, then pad 215 is used to apply hair removal solution 225 from second pouch 220. If pad 215 is initially permeated with the hair removal solution, then the additional hair removal solution 225 in second pouch 220 extends the coverage area for the hair removal system.

In yet another embodiment of the hair removal system, an applicator pad permeated with the hair removal solution is placed in first pouch 210. A cleaning pad permeated with a cleaning solution is placed in second pouch 220. The applicator pad is used to apply the hair removal solution to the skin and the cleaning pad is used to remove the hair and residue.

In the preferred embodiment of the hair removal solution, the hair removal solution comprises the following effective amounts of ingredients:

TABLE 1

| | % of solution (weight |
|---|---|
| INGREDIENTS | per volume (w/v)) |
| Demineralized Water | About 71.0 |
| Mineral Oil, U.S. Grade 100% | About 8.0 |
| Calcium Hydroxide, 100% concentration | About 3.0 |
| Cetearyl Alcohol, 100% concentration | About 6.0 |
| Ceteareth-20, 100% concentration | About 6.0 |
| Collagen, 90% concentration | About 1.0 |
| Tocopherol (Vitamin E), 100% concentration | About 0.001 |
| Calcium Thioglycolate, U.S. Grade 85% | About 2.0 |
| Lanolin, 100% concentration | About 2.0 |
| Fragrance | Quantity Sufficient |

The ingredients called for in each part are pre-mixed in the following manner. A first mixture is made by bringing an effective amount of demineralized water to about 75° C. and adding an effective amount of calcium hydroxide to the water as per Table 1. In other embodiments, other alkalis may be used in place of calcium hydroxide, such as sodium hydroxide. The first mixture is brought to about 75° C. to dissolve the ingredients and then agitated such that the calcium hydroxide particles are suspended in the liquid. The first mixture is maintained at 75° C.

A second mixture is made where cetearyl alcohol, cetereath-20, lanolin, and mineral oil in effective amounts are agitated in such a manner as to form an emulsion. The effective amounts of cetearyl alcohol, cetereath-20, lanolin and mineral oil are given in Table 1. The contents of the second mixture are then dissolved completely at a temperature of 65° C. to dissolve the ingredients. The second mixture is added to the first mixture to yield a third mixture. Demineralized water is added to bring the temperature of the third mixture down to about 30° C. in preparation for the next phases of the process.

A fourth mixture is made by mixing an effective amount of vitamin E and collagen, up to any temperature known in the art, such that each ingredient retains its chemical composition. An effective amount of vitamin E and collagen are given in Table 1. A fragrance may also be added as per preference. The fourth mixture is added to the third mixture to yield a fifth mixture.

A sixth mixture is made by adding amount of water to an effective amount of calcium thioglycolate such that the calcium thioglycolate is made soluble. In another embodiment, calcium thioglycolate may be replaced with sodium thioglycolate or other thioglycolic acid. The effective amount of thioglycolic acid is given in Table 1. The temperature of the sixth mixture is maintained at about 35° C. before it is added to the fifth mixture. The sixth mixture is added to the fifth mixture to yield the hair removal solution.

The manufacture and assembly of the hair removal system is now described. In one embodiment of the hair removal system, the edges of front and back pieces 116 and 118 are lined up and the perimeter of package 106 is sealed leak-tight on three sides creating the inner pouch between the front and back pieces 116 and 118. Pad 102 permeated with the hair removal solution is placed within the inner pouch. The fourth edge of the front and back pieces are then sealed together in a substantially leak-tight manner.

In another embodiment of the hair removal system, the pad is placed on a first sheet of material. The pad is sized smaller than the first sheet of material. The sheet of material is leak-proof and can be any material such as plastic, paper or combination thereof. Enough hair removal solution to cover the surface area and saturate the pores is applied to the pad. Alternatively, the pad is pre-soaked with the hair removal solution. A second sheet of material similar to the first sheet is then laid on top of the first sheet. The first and second sheets are then fused together to such that a pouch is formed in between the first and second sheets. The resultant pouch, with the enclosed pad, is sealed leak-tight.

In yet another embodiment of the hair removal system, the pad is placed on a first sheet of material. Similar to the previous embodiment, the pad is sized smaller than the first sheet of material. A second sheet of material similar to the first sheet of material is then laid on top of the first sheet. The first and second sheets are then fused together such that a first pouch is formed therein, the pad being contained within the first pouch. The resultant first pouch is sealed. A third sheet of material similar to the first and second sheets is laid on top of the first sheet of material. The first and third sheets are then fused together such that a second pouch is formed. A hair removal solution, or a cleaning pad, is then inserted into the second pouch. The second pouch is then sealed in a leak-tight manner.

The hair removal system may be used for removing unwanted hair on any part of the body. In one embodiment of the hair removal system, when a user obtains a package of the hair removal system, the user first identifies where the perforation is on the package. The user then uses the perforation to tear the package open. Once the package is opened, the user finds the pad contained within the package and removes the pad from the package. If the package contains a pad that is folded into a size that fits into the package, the user will then unfold the pad. The user contacts the pad to the area with unwanted hair. Once contact is made, the user may wipe the area such that the hair removal solution embedded within the pad is spread on the area containing unwanted hair. The hair removal solution is then left on the area for about ten minutes. A damp cloth is then used to remove the hair removal solution and the area is rinsed with warm water, followed by cold water. The area once containing undesired hair should be free of hair.

In another embodiment of the hair removal system, the package may include a first pouch containing the pad and a second pouch containing hair removal solution or a cleaning pad. The user opens the package at the perforation and removes the pad from the first pouch.

The user may apply the hair removal solution directly from the second pouch to the area containing unwanted hair, or the user may insert the pad into the second pouch. The user then wipes the hair removal solution in and around the area containing unwanted hair. The hair removal solution is left on the area for about ten minutes. A damp cloth is then used to remove the hair removal solution and the area is wiped with the cleaning pad or rinsed with warm water, followed by cold water. The area once containing undesired hair should be free of hair.

The hair removal system as described herein is a convenient, painless, non-irritating, compact, portable, disposable, easy-to-use, all-encompassing system for removing hair. The user simply opens the package, removes the pad, applies the hair removal solution for the prescribed time, and then removes the hair and residue with the cleaning pad or uses a towel or water. The hair removal system requires no further solutions, applicators, tools, or other paraphernalia for its application and use.

While the invention has been described with reference to preferred embodiments, it will be understood by those skilled in the art that various changes may be made and equivalents may be substituted for elements thereof without departing from the scope of the invention. In addition, many modifications may be made to adapt the teachings of the invention herein to a particular situation without departing from the essential scope thereof. Therefore, it is intended that the invention not be limited to the particular embodiments disclosed as the best mode contemplated for carrying out this invention, but that the invention will include all embodiments falling within the scope of the appended claims.

What is claimed is:

1. A system for hair removal, comprising:
a hair removal solution;
a first pad for applying the hair removal solution; and
a package enclosing the first pad.

2. The system of claim 1, wherein the first pad comprises a porous material.

3. The system of claim 1, wherein the first pad includes a plurality of pores to be impregnated with the hair removal solution.

4. The system of claim 1, wherein the package includes first and second pieces of material joined together to form a first inner pouch to enclose the first pad.

5. The system of claim 4, wherein the package includes a third piece of material joined together with the first piece of material to form a second inner pouch.

6. The system of claim 4, further including a portion of the hair removal solution enclosed in the second inner pouch.

7. The system of claim 4, further including a second pad enclosed in the second inner pouch.

8. The system of claim 1, wherein the package is sealed.

9. A hair removal system, comprising:
a hair removal solution;
a first pad for applying the hair removal solution; and
a package including a first piece of material and a second piece of material, wherein the first piece of material is joined to the second piece of material to form a first pouch for enclosing the first pad.

10. The hair removal system of claim 9, wherein the package further includes a third piece of material joined to the first piece of material to form a second pouch.

11. The hair removal system of claim 9, further including a portion of the hair removal solution enclosed in the second pouch.

12. The hair removal system of claim 9, further including a second pad enclosed in the second pouch.

13. The hair removal system of claim 9, wherein the first pad comprises a porous material.

14. The hair removal system of claim 9, wherein the first pad includes a plurality of pores to be impregnated with the hair removal solution.

15. A method for manufacturing a hair removal system, comprising:
providing a first pad within which to impregnate hair removal solution;
placing the first pad in a first pouch formed between first and second pieces of material; and
sealing the first pouch.

16. The method of claim 15 further including impregnating the first pad with the hair removal solution.

17. The method of claim 15, wherein the first pad comprises a porous material.

18. The method of claim 15, wherein the first pad includes a plurality of pores to be impregnated with the hair removal solution.

19. The method of claim 15 further including joining a third piece of material together with the first piece of material to form a second pouch.

20. The method of claim 19, further including enclosing a portion of the hair removal solution in the second pouch.

21. The method of claim 20, further including enclosing a second pad in the second pouch.

22. A method of manufacturing a hair removal solution, comprising:
preparing a first mixture by suspending an alkali in water and heating the first mixture to a first temperature;
preparing a second mixture by emulsifying cetearyl alcohol and cetereath- 20 and heating the second mixture to a second temperature;
adding the second mixture to the first mixture to yield a third mixture and bringing the third mixture to a third temperature;
preparing a fourth mixture by mixing water with thioglycolic acid and heating to a fifth temperature; and
adding the fourth mixture to the third mixture to yield the hair removal solution.

23. The method of claim 22, wherein the alkali is selected from a group consisting of calcium hydroxide and sodium hydroxide.

24. The method of claim 22, wherein the thioglycolic acid is selected from a group consisting of calcium thioglycolate and sodium thioglycolate.

25. The method of claim 22, further including adding mineral oil to the second mixture.

26. The method of claim 22, wherein the hair removal solution comprises:
about 71% weight per volume (w/v) water;
about 3% w/v alkali;
about 6% w/v cetearyl alcohol
about 6% w/v cetereath-20; and
about 2% w/v thioglycolic acid.
